(19) United States
(12) Patent Application Publication
(10) Pub. No.: US 2011/0106222 A1
(43) Pub. Date: May 5, 2011

(54) TREATMENT DEVICE FOR DELIVERING RADIATION TO AN EXTERNAL AREA OF THE HUMAN BODY

(76) Inventors: Brian Wilson, Toronto (CA); Brock Miller, Toronto (CA); Robert Weersink, Toronto (CA); Kai Zhang, Toronto (CA); George Newtchev, Toronto (CA)

(21) Appl. No.: 12/736,679

(22) PCT Filed: Apr. 24, 2009
(86) PCT No.: PCT/GB2009/050420

§ 371 Date: Jan. 18, 2011

(30) Foreign Application Priority Data
Apr. 30, 2008 (GB) 0807850.3

(51) Int. Cl. A61N 5/06

(52) U.S. Cl. 607/88

(57) ABSTRACT

A treatment device (2) includes a mobile cart (4) on which is mounted a housing (6) and an upright 8 to which is pivotally mounted, at hinge region (9), a second upright (11). An arm (12) is pivotally mounted at hinge region (10) to upright (11). Arm (12) supports a head piece (14) via a hinged joint (16). The housing (6) encloses a power supply, parts of a closed circuit cooling system arranged to cool the head piece (14) in use and electronic components. A user-operable control panel (18) for the device is mounted at a convenient height on the upright (8).

This invention relates to a treatment device and particularly, although not exclusively, relates to a radiation-emitting, for example light-emitting, device for use in activating a photosensitizing compound which may be used in therapy, for example photodynamic therapy (PDT). Preferred embodiments relate to treatment of human skin conditions and/or treatment of wounds in humans.

Applicant's co-pending application describes a range of photosensitizing compounds, for example drugs, for use in skin treatments, such as wound healing. In the presence of light and oxygen, such compounds can be activated to have a cytotoxic effect, for example against micro-organisms associated with a wound. Furthermore, the compounds also have a skin healing effect separate from any anti-microbial effect.

Known light emitting devices for use in PDT tend to be bulky and unergonomic. Furthermore, they need to be set up by highly skilled operatives, who must make various calculations and/or adjustments to deliver an appropriate light dosage. This makes it practical for treatments using the devices only to be carried out in hospitals and other central locations with appropriate resources.

It is an object of the present invention to address problems associated with treatment devices as aforesaid and generally.

The invention is based, in one embodiment, on the provision of a treatment device which is relatively compact and ergonomically designed such that it can be transported and used by non-skilled or semi-skilled operatives, with minimal training, to treat human skin conditions such as wounds.

According to a first aspect of the invention, there is provided a treatment device for delivering radiation to a treatment area, said device including radiation emitting means.

Said device is preferably for delivering radiation to a treatment area for treatment of a human skin condition and/or treatment of a wound in said area. Said skin condition may comprise a cosmetically undesirable skin condition and, accordingly, said device may be used in cosmetic treatments.

Said device is preferably for delivering radiation to an external area of a human body to treat said external area. Thus, said treatment area is preferably an external area of a human body. For example, it may be a leg, foot or arm. It may comprise the face and be used for example to treat acne.

Said device may be used to treat any condition where the integrity of tissue is damaged, including treatment of chronic or acute wounds. The device may be used to treat any treatment area wherein infection may be present and/or where it is desired to treat actual or potential infection.

Wounds which may be treated using said device include surgical wounds; bites; burns; acid and alkali burns; cold burn (frostbite), sun burn, minor cuts, major cuts, abrasions, lacerations, wounds caused by gunshot or knife injury; wounds caused by congenital disorders; wounds following surgery; periodontal disease; wounds following trauma; tumour associated wounds, which can be classified as malignant cutaneous ulcers related to the primary tumour or metastases; ulcers including diabetic ulcers; leg ulcers; foot ulcers; and pressure sores.

Acute wounds which are caused by external damage to skin include surgical wounds, bites, burns, cuts and abrasions, as well as more traumatic wounds such as lacerations and those caused by crush or gun shot injuries. Chronic wounds which are most frequently caused by endogenous mechanisms associated with a predisposing condition that ultimately compromises the integrity of dermal or epithelial tissue include leg ulcers, foot ulcers and pressure sores.

Said device may be used to treat inflammatory disorders of the skin that result in itching, crusting, scaling or blisters, such as eczema, psoriasis and acne.

Said device may be used to treat pre-malignant/malignant cutaneous conditions.

Said treatment device may be used to treat a wound or part of a wound of area up to 250 cm², for example up to 200 cm² or up to 100 cm².

Said radiation emitting means is preferably arranged to deliver light. The radiation emitting means may emit radiation in the range 400 nm to 900 nm. The peak output of the radiation emitting means may be in the range 500-700 nm, preferably in the range 600-700 nm more preferably in the range 650-700 nm, especially in the range 660-680 nm.

Said radiation emitting means may have a primary power at the surface of the treatment area of at least 25 mW/cm², suitably at least 50 mW/cm², preferably at least 75 mW/cm², more preferably at least 90 mW/cm². The primary power may be less than 400 mW/cm², preferably less than 300 mW/cm², more preferably less than 200 mW/cm².

Said radiation emitting means preferably comprises a multiplicity of light emitting diodes (LEDs). Said radiation emitting means may include in excess of 10, preferably in excess of 20, more preferably in excess of 25 individual LEDs per cm². The number may be less than 500, suitably less than 250, preferably less than 150, more preferably less than 75 individual LEDs per cm².

Said radiation emitting means may include a plurality of arrays of LEDs. The LEDs are preferably mounted on a support which is thermally conductive, suitably so that a cooling means can be arranged to remove heat from the support and therefore from the LEDs in use. The support is preferably substantially rigid.

Said radiation emitting means may comprise a first radiation emitting region and a second radiation emitting region. Said first and second regions may include respective arrays of LEDs. Said first and second regions are preferably movable, for example pivotable, relative to one another. A pivot axis is preferably defined between the first and second regions. Relative movement of the first and second regions is suitably arranged to allow the radiation emitting means to be positioned close to a treatment area so that the spacing of radiation emitting means (e.g. LEDs) from the treatment area is substantially constant.

Said first region is suitably pivotable through an angle of at least 10°, preferably at least 45°, more preferably at least 90°, especially at least 135° relative to the second region. A hinge means is preferably arranged between the first and second regions. It is preferred that the hinge means separates the first and second regions by the minimum distance so as to minimise and/or avoid “dark” areas which could be associated with any gap in radiation output between the first and second regions. The distance between the first and second regions may be less than 2 cm, preferably less than 1 cm, especially less than 0.5 cm.

Said first region may be substantially planar or have a small radius of curvature. In the latter case, it may be concave. Said second region may be substantially planar or have a small radius of curvature. In the latter case, it may be concave.

Said treatment device preferably includes facilitation means to facilitate pivotal movement of the first and second radiation emitting regions relative to one another. Said facilitation means may include an opening in which an operator's hand may be inserted, (suitably so that it extends across a hinge means between the first and second regions) wherein the opening is arranged so that an operator may flex his/her hand when present within the opening to cause said pivotal movement.

Said radiation emitting means may include a third radiation emitting region (which is preferably substantially planar) adjacent and/or contiguous with said second region (which is preferably substantially planar), wherein said third region may include an array of LEDs. Said second and third regions may not be movable (e.g. pivotable) relative to one another; they may be substantially immovably fixed in position relative to one another. Said third region may be angled relative to said second region (e.g. when viewed in cross-section).

Said radiation emitting means may include a fourth radiation emitting region (which is preferably substantially planar) adjacent and/or contiguous with said third region and suitably arranged on an opposite side of the third region to the position of said second region. Said third and fourth regions may not be movable (e.g. pivotable) relative to one another; they may be substantially immovably fixed in position relative to one another. Said fourth region may be angled relative to said third region.

Said second and fourth regions preferably extend away from said third region on the same side thereof. Together the first, third and fourth regions may define a trough shape.

Said radiation emitting means may include a fifth radiation emitting region (which is preferably substantially planar) adjacent and/or contiguous with said first region (which is preferably substantially planar), wherein said fifth region may include an array of LEDs. Said first and fifth regions may not be movable (e.g. pivotable) relative to one another; they may be substantially immovably fixed in position relative to one another. Said fifth region may be angled relative to said first region (e.g. when viewed in cross-section).

Said radiation emitting means may include a sixth radiation emitting region (which is preferably substantially planar) adjacent and/or contiguous with said fifth region and suitably arranged on an opposite side of the fifth region to the position of said first region. Said fifth and sixth regions may not be movable (e.g. pivotable) relative to one another; they may be substantially immovably fixed in position relative to one another. Said sixth region may be angled relative to said fifth region.

Said first and sixth regions preferably extend away from said fifth region on the same side thereof. Together the first, fifth and sixth regions define a trough shape.

In one embodiment, said radiation emitting means may include first, second and third radiation emitting regions, wherein a first hinge means is provided between the first and second regions; and a second hinge means is provided between the second and third regions.

Said radiation emitting means, for example LEDs, are preferably arranged substantially to face a treatment area in use. Thus, preferably a light guide (or the like) is not provided and arranged to deliver and/or deviate radiation between its point(s) of production and delivery to the treatment area. Said radiation emitting means are preferably arranged to be positioned very close to a treatment area in use. For example, the distance between the LEDs and a treatment area may be less than 5 cm, preferably less than 3 cm in use.

Said device may include a cover over the radiation emitting means for separating the radiation emitting means from a treatment area in use. The distance between the cover and the radiation emitting means may be in the range 0-30 mm, preferably 0-20 mm. The radiation emitting means are preferably arranged to direct radiation towards the cover and preferably primarily in a direction which is substantially perpendicular to a face of the cover.

Said cover may be arranged to diffuse radiation, suitably so that radiation output from the radiation emitting means passes through the cover and emerges as a substantially uniform output for impinging a treatment area.

Said cover may comprise a material which both covers the LEDs and extends between adjacent LEDs to some extent. For example, the cover may be formed using a curable material which is applied over the LEDs in such a way that it penetrates gaps between adjacent LEDs. The curable material may then be cured to define said cover. Said curable material is preferably a curable plastics material, for example a silicone.

Said treatment device preferably comprises a head member which preferably includes said radiation emitting means. Said head member suitably includes a radiation emitting face which suitably includes said first and second radiation emitting regions described above and, when provided, includes said third, fourth, fifth and/or sixth regions. Said radiation emitting face is suitably arranged to face a treatment area in use. The face may have an area in the range 2 cm² to 250 cm², preferably in the range 50 to 220 cm². Said face is suitably a component of a housing which comprises said radiation emitting means and, preferably, houses at least part of a cooling means.

The housing suitably incorporates hinge means as described above. Preferably, a surface of the housing which suitably faces in a generally opposite direction compared to that of said radiation emitting face is preferably substantially flat. It may be arranged to provide a position of location for an operator's hand as part of the facilitation means described. The head member may include an opening which may be defined on one side by said surface of said housing to define part of a region into which an operator's hand may be inserted in use. The opening may be defined in part by a collar which may be fixed to said surface of said housing. The head may have a depth of less than 350 mm. It may have a height of less than 300 mm. It may have a width of less than 250 mm.

The head member may include spacer means for spacing a light emitting surface (and/or the LEDs) from a treatment area in use. The spacer means may comprise one or more projections arranged around the periphery of the device, suitably outside an area within which the LEDs are arranged.

Said treatment device preferably includes a base which is suitably arranged to be seated on the ground in use. Said head member is preferably movable relative to the base. It is preferably movable upwards and downwards relative to the base. It is preferably pivotable relative to the base.

Advantageously, movable parts of the device, for example joints, hinges or pivots are arranged so that once moved to a new position, they remain in the new position. Thus, a head member of the device can be moved by an operator to a position adjacent a treatment area. Once the position has been reached an operator may disengage himself/herself from the device and the head member remains in the selected position. A supplementary locking means may be provided for locking the head member in the selected position. Said locking means may be arranged to be operated by the operator.

Said treatment means preferably includes cooling means for dissipating heat from the radiation means. The cooling means may be arranged to remove at least 5,000 Joules per minute preferably 10,000 Joules per minute, especially at least 12,500 Joules per minute from the radiation means. The cooling means may remove less than 30,000 Joules per minute, suitably less than 25,000 Joules per minute.

The cooling means is preferably arranged to remove heat by conduction of heat to a cooling fluid which may comprise or consist essentially of water. The cooling fluid is preferably arranged to flow towards and away from the radiation emitting means. The treatment device preferably includes a heat exchanger spaced from the radiation emitting means and arranged to remove heat from cooling fluid which has flowed from said radiation emitting means.

Pump means is suitably provided for delivering cooling fluid to and removing cooling fluid from the radiation emitting means.

The treatment device preferably includes a head member as described above, wherein at least part of said cooling means is arranged in the head member. In this regard, preferably cooling fluid is arranged to flow in the head member towards and away from radiation emitting means arranged in said head member. Said head member preferably incorporates conduit means through which cooling fluid may flow.

Said head member preferably includes a heat sink which is preferably substantially in thermal contact with the radiation emitting means. In this regard, the radiation emitting means, for example LEDs, may be associated with for example mounted on a thermally conductive, for example metal, member. The member may be thermally coupled, for example using a thermal adhesive, to a heat sink which may suitably comprise a metal member, for example of aluminium. Said cooling fluid is suitably arranged to withdraw heat from the metal member. To this end, said heat sink preferably includes conduit means whereby cooling fluid may flow within and/or across the heat sink to remove heat therefrom. Said conduit means may define a convoluted path for cooling fluid.

Said head member preferably includes means for releasably securing a fluid supply line for delivering cooling fluid to the head member. The head member preferably includes means for releasably securing a fluid supply line for removing cooling fluid from the head member.

When the radiation means includes a first radiation emitting region and a second radiation emitting region, separate heat sinks may be provided for each of said first and second regions. Preferably, cooling fluid for said separate heat sinks emanates from a single source (e.g. a single heat exchanger) and is returned to a single source (e.g. a single heat exchanger).

The treatment device preferably includes a head member (which suitably incorporates part of a said cooling means) and a heat exchanger (which suitably incorporates part of a said cooling means) which are suitably spaced from each other. A first conduit suitably extends from the heat exchanger to the head member wherein suitably said conduit is arranged to deliver cooling fluid to the head member. A second conduit suitably extends from the heat exchanger to the head member wherein suitably said conduit is arranged to withdraw fluid from the head member towards said heat exchanger. Said first conduit suitably is associated with, for example is enclosed by, a support means of the treatment device which support means extends between the heat exchanger and the head member. Similarly, said second conduit suitably is associated with, for example is enclosed by, said support means of the treatment device. Said support means is preferably articulated and/or pivotable to enable the head member to be positioned adjacent a treatment area in use.

The support means may include an upright and an arm which is pivotable relative to the upright. The head member may be fixed to said arm. It may be pivotably mounted thereon.

Said head member is preferably arranged to be detached from other parts of the treatment device, for example to enable the head member to be removed. Removal of a head member may be desirable if a head member is broken and needs repairing; or if an alternative size and/or configuration of head member is desired to be used in a treatment. The treatment device is suitably arranged so removal of a head member involves breaking electrical and/or fluid connections between the head member and other parts of the device.

Said head member may include control means for controlling operation of the head, e.g. operation of the radiation means. The head may include a display means for displaying information relevant to operation of the device.

Said treatment device may have a weight of less than 50 kg, preferably less than 40 kg, more preferably less than 30 kg.

According to a second aspect of the invention, there is provided a kit comprising a treatment device according to the first aspect which includes a first head member as described according to the first aspect and a second head member, wherein said first and second head members are different for delivering radiation to treatment areas of different sizes, shapes or positions.

According to a third aspect of the invention, there is provided a method of treating a treatment area, for example for medical or cosmetic treatment of a human skin condition, the method comprising:

(i) contacting a treatment area with a photosensitizer compound; and

(ii) delivering radiation to said treatment area using a treatment device as described according to the first aspect in order to activate the photosensitizer compound.

Said photosensitizer may be belong to any of the following structural classes:

1. Porphyrins. Some specific examples are Photofrin.®; 3,1-meso tetrakis (o-propionamido phenyl) porphyrin; hematoporphyrin (HP); protoporphyrin; uroporphyrin 2. Chlorins, particularly tetra(hydroxyphenyl)chlorines and o-substituted tetraphenyl porphyrins (picket fence porphyrins). Some specific examples are Foscan.®, chlorin-e6; mono-1-aspartyl derivative of chlorin-e6.

3. Bacteriochlorins, particularly tetra(hydroxyphenyl)-bacterio-chlorins

4. Bacteriochlorophylls

5. Pheophorbides

6. Synthetic diporphyrins and dichlorins

7. Phthalocyanines, for example sulfonated derivatives of zinc phthalocyanine, sulphonated derivatives of chloraluminum phthalocyanine and hydroxyaluminium phthalocyanine; ring-substituted cationic phthalocyanines.

8. Naphthalocyanines

9. Verdins

10. Purpurins. Specifc examples are tin and zinc derivatives of octaethylpurpurin (NT2), and etiopurpurin (ET2)

11. Benzoporphyrin derivatives (BPD), for example, benzoporphyrin monoacid derivatives, tetracyanoethylene adducts of benzoporphyrin, dimethyl acetylenedicarboxylate adducts of benzoporphyrin, Diels-Alder adducts, and monoacid ring “a” derivative of benzoporphyrin

12. Low density lipoprotein mediated localization parameters similar to those observed with hematoporphyrin derivative (HPD)

13. Texaphyrines, and non-tetrapyrrole porphyrins

14. Anthracenediones, e.g. aminoanthraquinones

15. Anthrapyrazoles

16. Phenoxazinium and phenoxazone dyes

17. Phenothiazinium dyes. Some specific examples are Methylene blue, New Methylene blue and Toluidine blue and phenothiazone dyes.

18. Phenoselenazinium dyes

19. Phenotellurazinium dyes

20. Acridinium compounds

21. Fluoresceins, especially halogenated fluoresceins. Some specific examples are Erythrosin, Rose Bengal and Eosin.

22. Cyanine dyes

23. Squarylium dyes

24. Triarylmethine and diarylmethine cationic dyes

25. Chalcogenapyrylium dyes

26. Cationic selena- and tellura-pyrylium derivatives 27. 5-Amino levulinic acid and its alkyl esters. Some specific examples of the latter are methyl 5-aminolevulinate and n-hexyl 5-aminolevulinate.

The photosensitizer may have a maximum absorption in the range 400 nm to 900 nm, preferably in the range 500 to 700 nm, more preferably in the range 600 to 700 nm.

An especially preferred class of photosensitizers is the phenothiazinium class. Preferred phenothiaziniums may be of formula

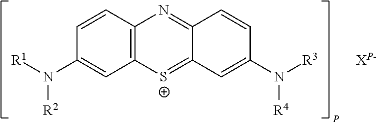

wherein R¹, R², R³ and R⁴ independently represent an optionally substituted linear, branched or cyclic hydrocarbon or R¹ and R² together with the N atom to which they are bonded form an optionally-substituted 5-, 6-, or 7-membered ring; and

X^{P-} is a counteranion and P is 1, 2 or 3.

Phenothiaziniums may be as described in US2004/0147508, the content of which is incorporated herein by reference.

Preferably, in preferred compounds R¹, R², R³ and R⁴ are the same and represent n-propyl, n-butyl, iso-butyl, n-pentyl, iso-pentyl, n-heptyl or n-hexyl. More preferably, R¹, R², R³ and R⁴ are the same and are selected from n-butyl and n-pentyl.

The compounds of Formula I may be formulated into a variety of pharmaceutical compositions which contain the compounds and pharmaceutically acceptable carriers, excipients, adjuvants (each selected for certain characteristics that permit optimal formulation of a pharmaceutical composition). The compositions include lipsomes, nano-particles, colloidal suspensions, micelles, microemulsions, vesicles and nanospheres.

The compositions may also comprise further components such as conventional delivery vehicles and excipients including solvents such as alcohols (for example ethanol, polyethylene glycol or n-butanol), dimethyl sulphoxide, water, saline, solubilisers such as castor oil derivatives for example ethoxylated castor oils like Cremophor EL (trade mark BASF AG) or Tween (trade mark, ICI Americas Inc) types, isotonising agents such as urea, glycerol, aminoethanol, propylene glycol, pH regulators, dyes, gelling agents, thickeners, buffers, and combinations thereof.

The concentration of photosensitizers used may be in the range 0.005 to 0.5 wt %. In some treatments for example in treatment of acne, a concentration of up to 5 wt % may be used.

Said composition is preferably for topical use. It may be delivered via a spray, lotion, suspension, emulsion, gel, ointment, salve, stick, soap, liquid aerosol, powder aerosol, drop or paste.

The method may include selecting a treatment area, contacting the treatment area with a said photosensitizer and operating the radiation emitting means to activate the photosensitizer.

The method may include operating a positioning or aiming mode of the device to facilitate positioning of the device. The mode may involve the light emitting means emitting radiation at a lower level (e.g. 5 to 20%, for example less than 10% of the maximum). In the mode, suitably, the light emitting means is operated at less than 20 mW/cm², preferably at less than 15 mW/cm² and, more preferably at 10 mW/cm² or less.

The device, in particular a head member thereof may be positioned by an operator manipulating the head member into position adjacent a wound. The head member is suitably manipulated and/or any spacing means positioned so that the head member conforms as closely as possible to the wound and/or the light emitting means are a constant distance from the surface of the wound. Once in position, the head member suitably remains in position after an operator has ceased to contact the head member. The method may include operation of a locking means, preferably by an operator, to lock the head member in position. Such a locking means suitably supplements another means associated with said device by which the head member can remain in position after an operator has ceased to contact the head member.

The device may be operated to deliver radiation to the treatment area for a suitable period of time which may be from one minute up to 5 hours, preferably 1 minute to one hour.

During operation of the device, said cooling means is preferably operated, suitably substantially continuously. This may ensure that the temperature of an outer face of the device which is positioned closest to a treatment area does not exceed 50° C., preferably does not exceed 45° C. and more preferably is no greater than about 40° C.

According to a fourth aspect of the invention, there is provided a combination for use in a medical or cosmetic treatment of a human skin condition, wherein said combination comprises a treatment device as described according to the first aspect and a photosensitizer compound.

Any feature of any aspect of any invention or embodiment described herein may be combined with any feature of any aspect of any other invention or embodiment described herein mutatis mutandis.

Specific embodiments of the invention will now be described, by way of example, with reference to the accompanying drawings, in which:

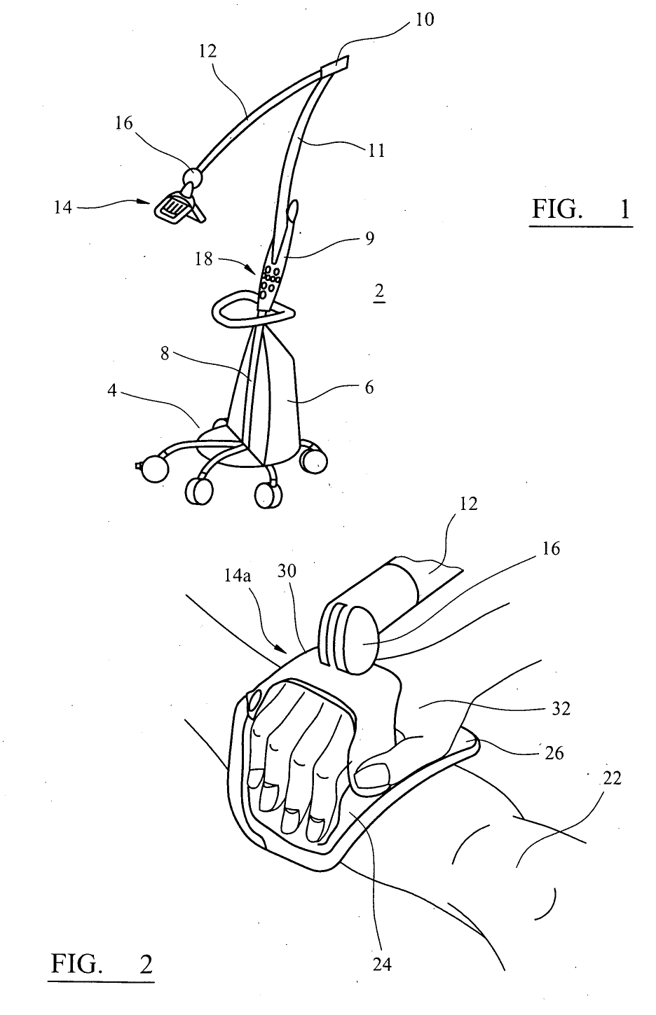

FIG. 1 is a perspective view of a light-emitting treatment device;

FIG. 2 is a perspective view showing a head piece in situ covering a wound on a patient's forearm;

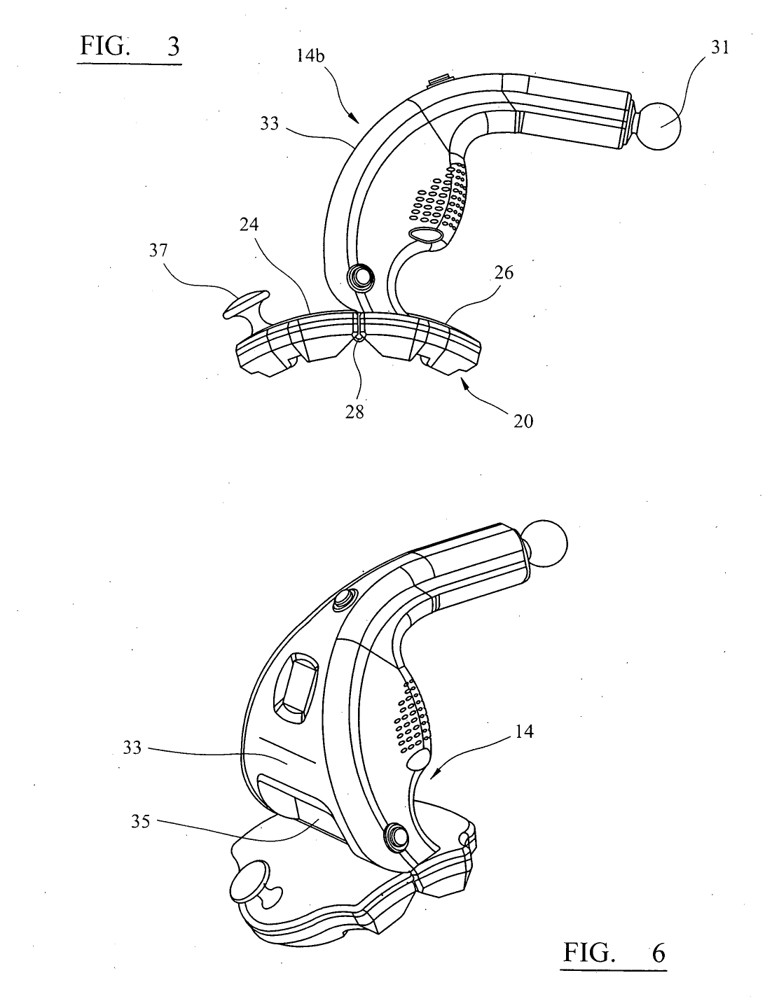

FIG. 3 is a side view of a head piece which is modified compared to the head piece of FIG. 2;

FIG. 4 is a front view of the head piece of FIG. 3;

FIG. 5 is a back view of the head piece of FIG. 3;

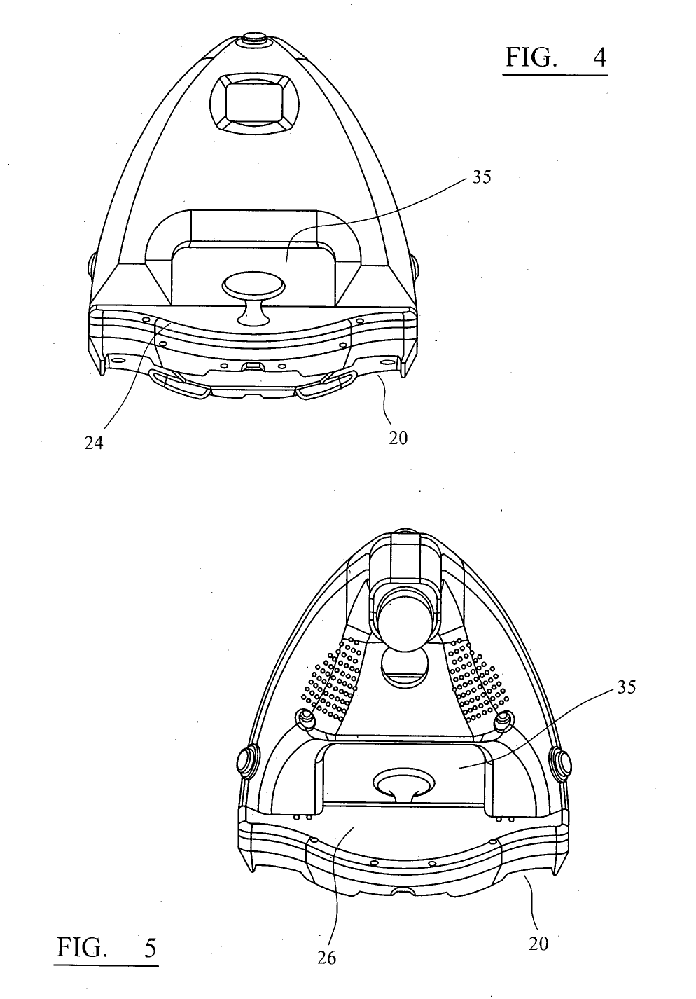

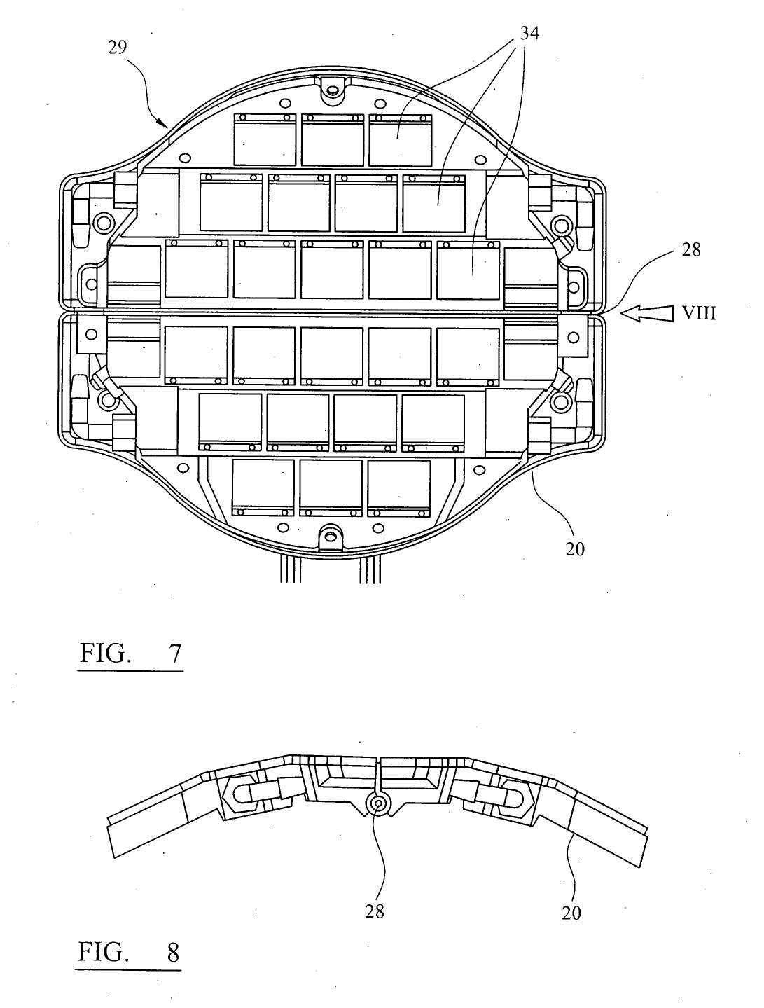

FIG. 6 is a perspective view of the head piece of FIG. 3;

FIG. 7 is an underneath plan view of an LED-containing assembly of the head piece of FIGS. 3 to 6;

FIG. 8 is a view of the assembly in the direction of arrow VIII in FIG. 7 but with an outer face of the head piece cut away;

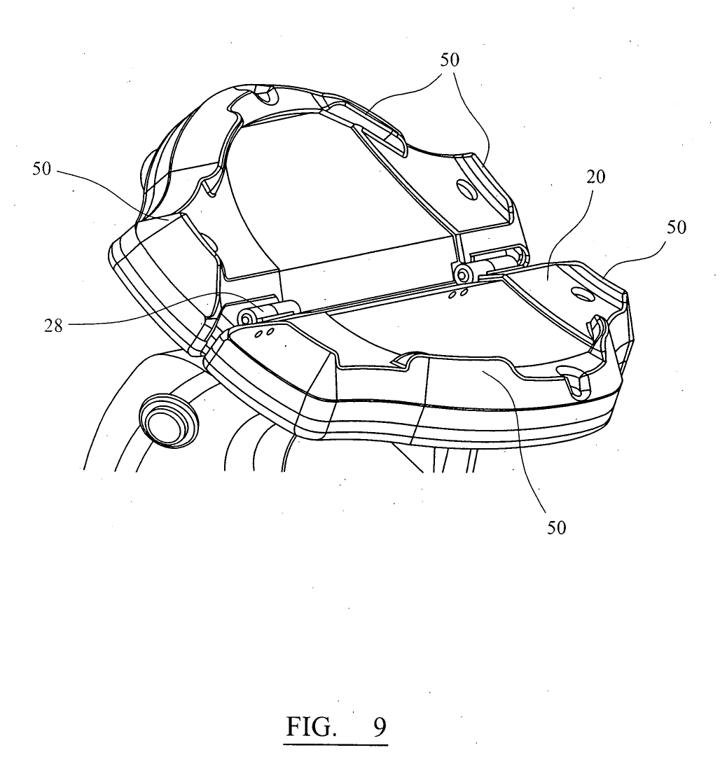

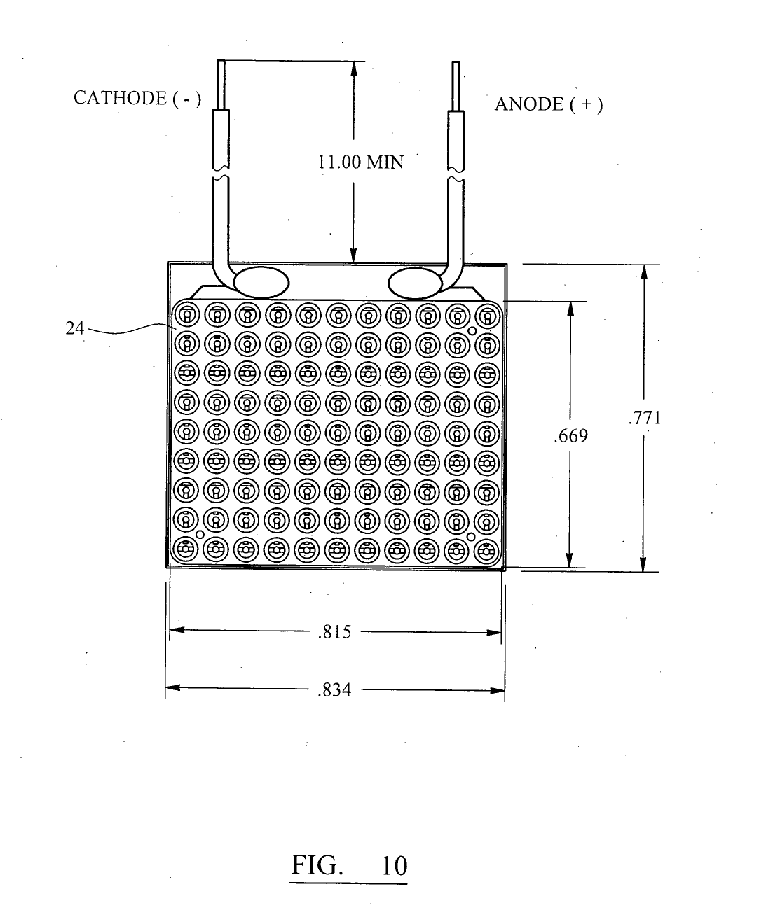

FIG. 9 is an underneath perspective view of the head piece of FIG. 3 with the LED assembly of FIG. 7 omitted;

FIG. 10 is an underneath plan view of an array of LED devices;

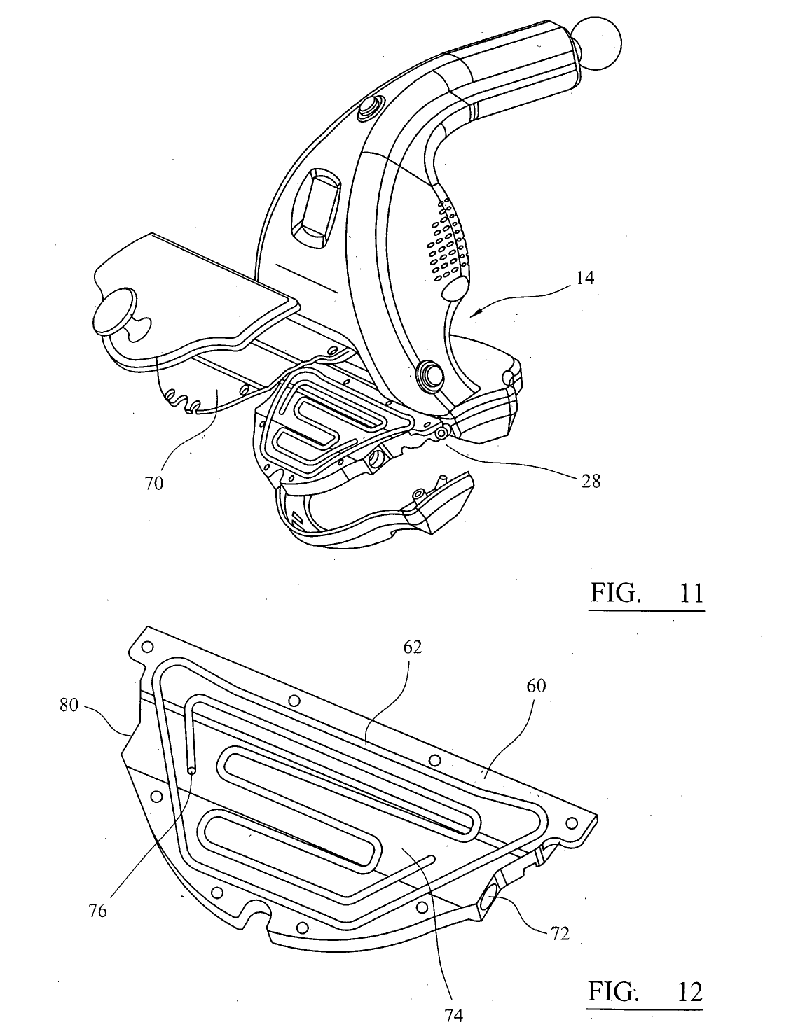

FIG. 11 is an exploded view of arrangement of FIG. 6, showing features of a cooling system;

FIG. 12 is an enlarged view of one panel of the cooling system;

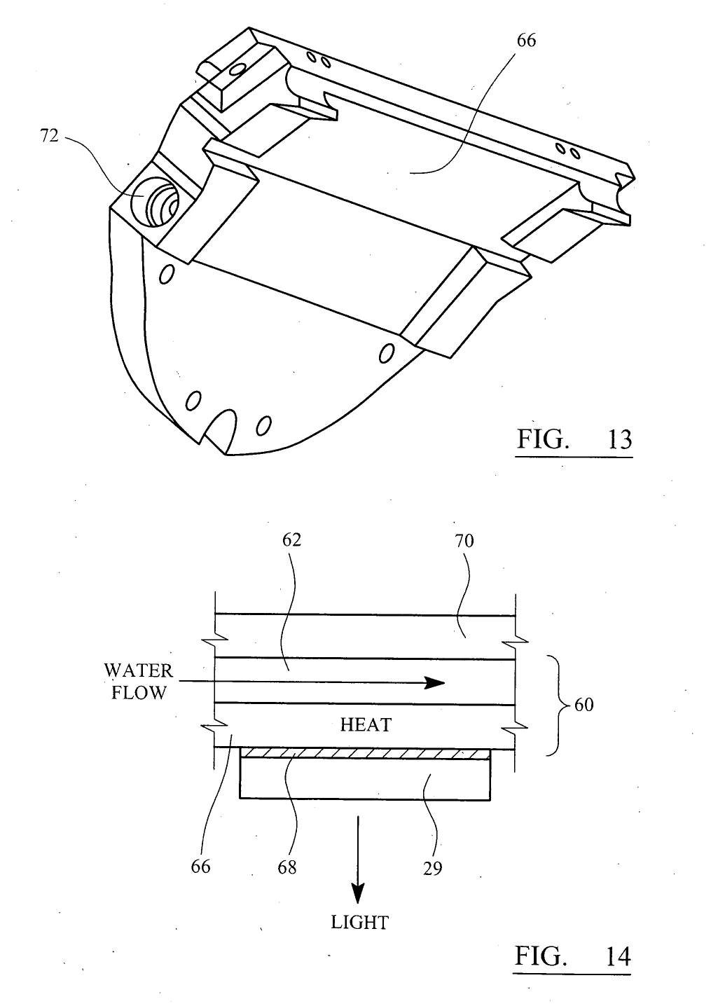

FIG. 13 is an underside view of a head piece with an LED assembly omitted;

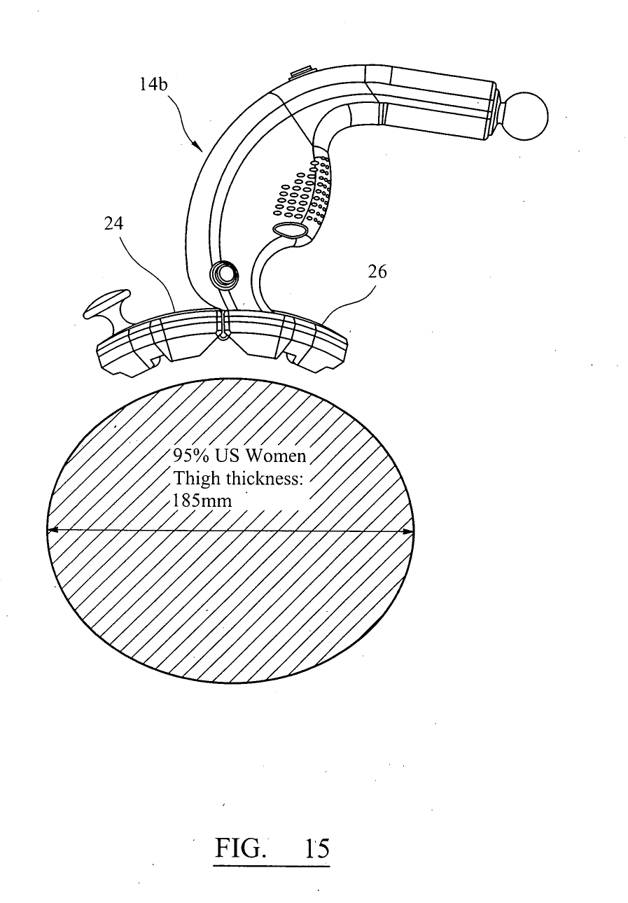

FIG. 14 is a schematic cross-section of the cooling system;

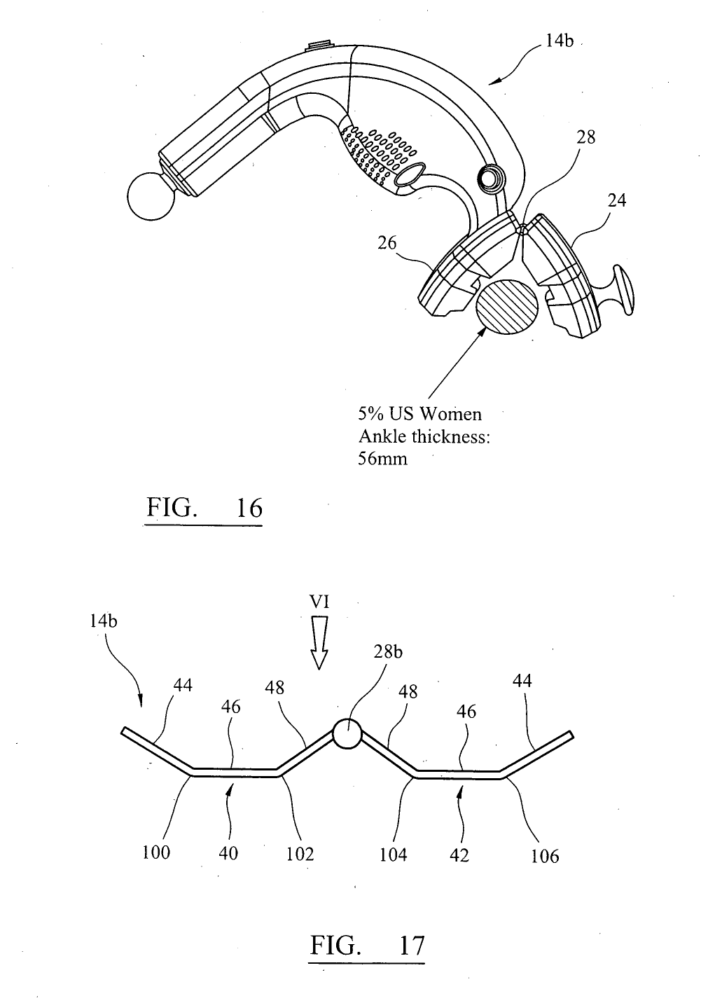

FIGS. 15 and 16 are side elevations showing the head piece of FIG. 3 associated with body parts of different radii of curvature;

FIG. 17 is a schematic cross-section of a head piece, to illustrate angling between lines of LED arrays of the head piece;

In the figures, the same or similar parts are annotated with the same reference numerals.

Referring to FIG. 1, the device 2 includes a mobile cart on which is mounted a housing 6 and an upright 8 to which is pivotally mounted, at hinge region 9, a second upright 11. An arm 12 is pivotally mounted at hinge region 10 to upright 11. Arm 12 supports a head piece 14 via a hinged joint 16. The housing 6 encloses a power supply, parts of a closed circuit cooling system arranged to cool the head piece 14 in use and electronic components. A user-operable control panel 18 for the device is mounted at a convenient height on the upright 8.

In use, the device is arranged to deliver light in a controlled and/or predetermined manner from head piece 14 to a wound to which has been applied a photosensitizer, as shown in FIG. 2.

The device and its operation are described in detail below.

Head pieces 14a (FIGS. 1 and 2) and 14b (FIGS. 3 to 16) are arranged to conform to a surface of skin which includes a wound (or other lesion) to be treated and apply light to the skin to activate a photosensitizer pre-applied to the wound. Referring to FIGS. 1 to 2, head piece 14a is pivotably mounted on arm 12 via hinged joint 16 thereby to allow the head piece to be arranged so that a light-emitting face 20 thereof can be positioned adjacent skin of a body part 22 which includes a wound (not shown). Head piece 14b includes a ball 31 (FIG. 3) by which it is pivotably mounted on an arm 12 (not shown in FIG. 3). The head piece 14a includes two panels 24, 26 which are pivotably mounted via a hinge so that the panels can be pivoted relative to one another thereby to adjust the angle between panels 24, 26 and thereby adjust the shape defined by the light emitting face 20. Similarly, head piece 14b include a hinge 28 which allows panels 24, 26 thereof to be pivoted relative to one another. As a result of pivoting of panels 24, 26 the shape of the light emitting face may be adjusted so that it conforms as far as possible to the shape of a body part to be treated using the device.

The head pieces are arranged so that they can easily be manipulated by an operator into position. In this regard, upwardly facing surfaces of the panels 24, 26 of the FIG. 2 embodiment are substantially flat and together with a bridging piece 30 define an opening which may comfortably receive a hand 32 of an operator. When so disposed, the operator can cause panels 24, 26 to pivot by flexing his/her hand thereby to cause the panels to adopt a desired configuration. The embodiment of FIG. 3 et seq comprises a bridging arrangement 33 which, together with panels 24, 26, defines an opening 35 which is arranged to receive an operator's hand as in the FIG. 2 embodiment. In addition, a knob 37 is provided as a further means whereby an operator can cause panels 24, 26 to pivot to define a desired configuration.

The hinge 28, hinged joint 16 (or ball 31 and socket (not shown), hinge region 10 and hinge region 9 of the devices are arranged such that once the head piece has been positioned adjacent a wound, it remains in position. Thus, it will be appreciated that it takes little skill or dexterity to position the head piece in a desired position over a wound.

The head pieces 14 incorporate arrays 34 of light-emitting diodes (LEDs) (although they are only shown in detail in the context of the FIG. 3 et seq embodiment) which are arranged to direct light outwardly away from light emitting face 20 of the device. The LEDs are suitably close together, even across the hinge 28, so that light output from face 20 is of substantially constant intensity across the face 20. The array of LEDs may be provided with respective covers (one on each side of hinge 28) to protect the LEDs and avoid direct contact between the LEDs and a wound being treated. Such covers may be arranged to be light scattering (e.g. they may be diffusing rather than transparent) thereby to improve the uniformity of light output from face 20. In one embodiment, the cover may comprise a curable medical grade silicone. Such a material, in an uncured state, may be applied directly on the LEDs to fill in gaps between adjacent LEDs. The outer surface of the material may be treated to define a smooth surface. On curing, the silicone defines a diffusing cover having a smooth washable outer surface.

Each array 34 of LEDs includes 99 separate LED devices 35 (FIG. 10) mounted on a metal support and arranged to define a rectangular array of dimensions about 1.8 cm by 2.1 cm. The arrays are mounted adjacent one another thereby to define a substantially continuous arrangement of LEDs across the face 20. Collectively, the LEDs have an output of 100 mW/cm². Such an output represents a compromise between being able to deliver a satisfactory quantity (in terms of that needed to activate a photosensitizer used) of light over a measurable time whilst not generating too much heat which could heat the wound detrimentally and/or will need to be conducted away using a cooling device as described hereinafter.

The LEDs have a peak wavelength of 670 nm±30 nm which may advantageously be used with preferred photosensitizers.

Wounds may be treated using the device by a relatively non-skilled or semi-skilled operator. In this regard, a photosensitizer can be applied to the wound in a predetermined manner. A preferred photosensitizer is a phenothiazine which may be applied using a formulation comprising 0.025 wt % of active ingredient at a rate of 15 microlitres/cm² of wound area. Then, the device 2 is manipulated so the head piece 14 is superimposed upon the wound. A face 20 of the head piece may contact the wound or the face may be spaced slightly from the wound using a spacer as described hereinafter. In any case, the device is precalibrated to deliver appropriate levels of light to the wound, in use. When the head piece is suitably positioned, the LEDs may be operated by the operator simply activating a button on control panel 18 (or on head piece 14 itself). The LEDs then operate for a period of time which is predetermined and factory programmed into device 2. The time is calculated according to the photosensitizer used, the power of the LEDs and the position of the head piece (i.e. the distance of the LEDs from the wound). However, it should be appreciated that the operator does not himself/herself determine the length of time or any other significant variable for use in the treatment. Accordingly, the operator need not exercise any significant skill in treating a wound in an appropriate and effective manner.

As described above, predetermining the distance of the LEDs from a wound is a factor in determining how long a wound should be exposed to light and/or for ensuring consistency in application of light to wounds. In one embodiment, a plastic film may simply be applied over a wound to which a photosensitizer has been applied and then the head piece may directly contact the plastic film in use. In the embodiment of FIG. 3 et seq, as shown particularly in FIG. 9, the face 20 of the head piece incorporates spacers 50, around the LEDs, at the periphery of the head piece. The spacers project from face 20 and are arranged to contact an area around a wound in use. As an alternative to individual spacers which project, a spacer may be defined by an endless ledge around the LEDs. In both cases, the spacer(s) is/are arranged to set the distance between the LEDs and the surface of the wound and thereby ensure a reproducible light dose (optical power density, Wcm⁻²) is delivered to the wound.

In some embodiments, the face 20 of the head piece may include a disposable protective cover which may contact a wound in use and be discarded afterwards to avoid the risk of the spread of infection between patients.

It will be appreciated that the LEDs generate heat and that is important to restrict the amount of heat passing into a wound since it is undersirable to heat the wound to any appreciable degree. It is preferred that face 20 of the head piece does not exceed about 40° C. To achieve this, the head piece includes a water cooling system as illustrated in FIGS. 11 to 14. The cooling system includes, on each side of the hinge 28, a respective aluminium body 60 in which a track 62 is defined in an upwardly facing face 64 thereof. LED assembly 29 (FIGS. 7 and 14) contacts lower face 66 and is secured thereto by a layer 68 of thermal adhesive, the arrangement being such that heat can be conducted from the LEDs, via the metal support on which they are mounted, via the thermal adhesive and into body 60. The body 60 is covered by an aluminium top plate 70.

Body 60 includes a water inlet 72 which is arranged to allow water to be delivered to a first end 74 of the track 62. The track traverses the body 60 in a convoluted manner and includes a second end 76 which communicates with an outlet at position 80 on the body 60. On the other side of hinge 28, the cooling system is a mirror image of the arrangement desired.

Water is delivered to the head piece from the housing 6 (FIG. 1) via a first conduit which passes through uprights 8, 11 and arm 12. In a region close to the head piece, the first conduit is bifurcated and separate conduits are connected to respective inlets 72 in bodies 60 arranged on opposing sides of the hinge 28.

Water is removed from the head piece via respective conduits which communicate with outlets of track 62 on opposing sides of hinge 28 and then feed into a second conduit which passes through uprights 8, 11 and arm 12 and returns to housing 6.

Housing 6 includes a heat exchanger and pump and is thereby arranged to deliver cool water to the head piece and remove water heated by heat conducted to it from the LEDs. The heat exchanger then cools the heated water and recirculates it.

Variations upon and/or additions to the device 2 described above are described below.

In one embodiment, the device 2 may be adapted to include a means for determining if face 20 is correctly positioned to delivery light to a wound, before the LEDs are operated at full power. In this regard, the LEDs may be arranged to be operated at low power, for example about 10% of the normal treatment power such as by the operator simply selecting a “positioning” or “aiming” mode of the device. In use, an operator may position the face 20 close to a wound but spaced slightly therefrom; the low power (positioning/aiming) mode of the LEDs may be selected; and the operator may then observe the wound and assess whether light from the LEDs is being satisfactorily directed at it. The head piece may then be repositioned if necessary. When the head piece appears to be optimally positioned, it may be moved to its operative position (e.g. abutting or slightly spaced from a wound surface) and the LEDs operated at full power.

The head piece 14b may be used in a number of ways to treat wounds in different locations on a patient. In one case, only one of panels 24, 26 may be superimposed on a wound, for example if the wound has a small area. In other embodiments, the wound may be positioned between panels 24, 26. In one case, respective panels 48 may be positioned substantially in line with one another across the hinge 28 so that the light emitting face 20 has a large radius of curvature and may contact a thigh (or the like) as shown in FIG. 15. In another case, the respective panels 24, 26 may be pivoted so that an acute angle is defined between them and consequently the light emitting face defines a configuration which may contact a body part (e.g. an ankle) having a small radius of curvature as illustrated in FIG. 16.

It should be appreciated that, in the FIG. 3 et seq embodiment, lines of LEDs are arranged on planar faces and the planar faces are angled relative to one another. This arrangement is exaggerated in FIG. 17. Referring to the figure, the head piece 14 includes first and second halves 40, 42 which are pivotably mounted via hinge 28b. Each half 40, 42 is made up of three panels 44, 46, 48 which are substantially planar and which incorporate arrays of LEDs 34. Each half comprises panels which are fixed in position relative to each other panel in the same half. Panel 44 defines an obtuse angle to panel 46; and panel 48 defines an obtuse angle to panel 46.

Further alternative head pieces may be provided, for example, head pieces may include multiple hinges to allow greater conformity with a body part. For example, hinges could be provided at one or more of points 100, 102, 104, 106 of FIG. 17. In addition, larger or smaller head pieces housing configurations as described herein may be provided.

In order to allow a wide range of patients and wounds to be treated, the radiation device may be arranged so that head pieces are interchangeable. Thus, apparatus may incorporate a mobile cart 4 as described in FIG. 1 and a plurality of different head pieces. The head pieces may have different sizes and/or different configurations. Changing a head piece is relatively straight forward. Firstly, water conduits of the cooling system are detached as are any electrical connections. Secondly, the head piece is detached from hinged joint 16. Thirdly, a new head piece is selected and the water conduits and electrical connections are made.

1. A treatment device for delivering radiation to a treatment area, the device including radiation emitting means.

2. A device according to claim 1, wherein the peak output of the radiation means is in the range 500 to 700 nm and said radiation emitting means has a primary power at a surface of the treatment area of at least 25 mW/cm² and less than 400 MW/cm².

3. A device according to claim 1, wherein said radiation emitting means comprises a multiplicity of light emitting diodes (LEDs) and includes in excess of 10 LEDs per cm².

4. A device according to claim 1, which includes a plurality of arrays of LEDs which are mounted on a support which is thermally conductive.

5. A device according to claim 1, wherein said radiation emitting means comprises a first radiation emitting region and a second radiation emitting region, wherein said first and second regions include respective arrays of LEDs and said first and second regions are movable relative to one another.

6. A device according to claim 5, wherein said radiation emitting means includes a third radiation emitting region adjacent and/or contiguous with said second region, wherein said third region includes an array of LEDs.

7. A device according to claim 5, wherein said radiation emitting means includes a further radiation emitting region adjacent and/or contiguous with said first region, wherein said further region includes an array of LEDs.

8. A device according to claim 6, wherein said first and third regions and, when provided, the further radiation emitting region are not movable relative to one another.

9. A device according to claim 1, which includes a head member which includes said radiation emitting means, said head member including a radiation emitting face which includes first and second radiation emitting regions wherein said face is a component of a housing which comprises said radiation emitting means.

10. A device according to claim 9, wherein said radiation emitting face has an area in the range 2 cm² to 250 cm² and said housing includes at least part of a cooling means.

11. A device according to claim 9, wherein a surface of the housing which faces in a generally opposite direction compared to that of said radiation emitting face is arranged to provide a position of location for an operators hand as part of a facilitation means which is arranged to facilitate pivotal movement of the first and second radiation emitting regions relative to one another.

12. A device according to claim 1, which includes a head member which includes spacer means for spacing a light emitting surface from a treatment area in use.

13. A device according to claim 1, wherein said treatment device includes a base which is arranged to be seated on a surface in use, a head member which is movable upwards and downwards relative to the base and also is pivotable relative to the base.

14. A device according to claim 1, wherein said treatment means includes cooling means for dissipating heat from the radiation means, said cooling means being arranged to remove at least 5000 Joules per minute.

15. A device according to claim 1, which includes a head member which includes at least part of a cooling means for dissipating heat from the radiation emitting means.

16. A device according to claim 15, wherein said head member includes a heat sink which is substantially in thermal contact with the radiation emitting means and a cooling fluid is arranged to withdraw heat from the heat sink.

17. A treatment device according to claim 1, which includes a head member which incorporates radiation emitting means, part of a cooling means and a heat exchanger, the head member and heat exchanger being spaced from one another, wherein a first conduit extends from the heat exchanger to the head member to deliver cooling fluid to the head member.

18. A device according to claim 12, wherein said head member is arranged to be detached from other parts of the treatment device.

19. A device according to claim 1, wherein the device is arranged to be operated in a positioning or aiming mode, wherein light emitting means of the radiation emitting means is arranged to emit radiation at a lower level compared to a level used in delivering radiation to a treatment area to effect a treatment.

20. A kit comprising a treatment device according to claim 1 which includes a first head member which includes radiation emitting means and a second head member which includes radiation emitting means, wherein said first and second head members are different for delivering radiation to treatment areas of different sizes, shapes or positions and said first and second head members are releasably securable to the treatment device.

21. A method of treating a treatment area, the method comprising
(i) contacting a treatment area with a photosensitiser compound; and
(ii) delivering radiation to said treatment area using a treatment device as described in claim 1 in order to activate the photosensitiser compound.

22. A method according to claim 21, which includes operating a positioning or aiming mode of the device to facilitate positioning of the device, prior to delivery of radiation in order to activate the photosensitiser compound.

23. A method according to claim 21, wherein a head member of the device is positioned by an operator manipulating the head member into position adjacent a treatment area, the head member being manipulated so that it conforms to the treatment area.

24. Use of a treatment device according to claim 1, for delivering radiation to human skin to treat a treatment area which has been contacted with a photosensitiser compound.

25. A combination for use in a medical or cosmetic treatment of a human skin condition, wherein said combination comprises a treatment device as described in claim 1 and a photosensitiser compound.
